(19) United States
(12) Patent Application Publication
(10) Pub. No.: US 2026/0041876 A1
(43) Pub. Date: Feb. 12, 2026

(54) SENSORY APPARATUSES, SYSTEMS, AND METHODS

(71) Applicant: Eliana Jennifer Wydra, Great Neck, NY (US)

(72) Inventor: Eliana Jennifer Wydra, Great Neck, NY (US)

(21) Appl. No.: 19/103,048

(22) PCT Filed: Aug. 14, 2023
(86) PCT No.: PCT/US2023/030132

§ 371 Date: Feb. 11, 2025

Related U.S. Application Data
(60) Provisional application No. 63/397,585, filed on Aug. 12, 2022.

(51) Int. Cl. A61M 21/02; A61M 21/00

(52) U.S. Cl. CPC A61M 21/02; A61M 2021/0022; A61M 2021/0044; A61M 2210/04

(57) ABSTRACT

The present technology relates to sensory apparatuses that are useful for individuals who need assistance with neurodevelopmental conditions such as autism spectrum disorder (ASD), sensory processing disorder (SPD), attention deficit disorder (ADD) and attention deficit hyperactivity disorder (ADHD). Such apparatuses can include a top cover connected to or integral with a top base plate, and a bottom cover connected to or integral with a bottom base plate, about which projections or wings having different visual or tactile properties, such as different textures, are rotatably attached. The present technology is also directed to systems and methods for the management of a neurodevelopmental condition.

### BACKGROUND

The present disclosure relates to exemplary embodiments of a sensory apparatus, and more particularly, to exemplary embodiments of a sensory apparatus having projections, also referred to herein as “wings,”that can have different textures.

Neurodevelopmental conditions such as autism spectrum disorder (ASD), sensory processing disorder (SPD), attention deficit disorder (ADD) and attention deficit hyperactivity disorder (ADHD) affect millions of people worldwide, including both children and adults. Through interventions such as therapy and directed sensory inputs, management of such conditions in affected individuals through the use of apparatuses and devices can help them gain social, coping, and communication skills.

Apparatuses and devices such as toys, gadgets, fidgets and the like that assist an affected individual in a visual or tactile way can be helpful tools in stimulating and enabling a user to practice focus, attention, calming, active listening, and a host of other positive and beneficial behaviors. Users have the opportunity to practice and reinforce learned behavioral and coping mechanisms by manipulating or otherwise engaging with such self-regulation devices. Such activities and interactions can lead to better management of neurodevelopmental conditions.

An ongoing need exists for apparatuses and devices that accomplish these goals, and that are also convenient and easy to transport, use and store. In particular, a sensory apparatus that can be carried around easily and used while going about activities of daily life, such as on a keychain, can be particularly useful.

### SUMMARY OF THE DISCLOSED TECHNOLOGY

In certain embodiments, the present technology is directed to apparatus configured to stimulate a tactile response in a user, the apparatus comprising:
(a) a top cover having a generally rounded top surface and an opposite surface connected to or integral with a top base plate;
(b) a bottom cover disposed opposite the top cover, having a generally rounded bottom surface and an opposite surface connected to or integral with a bottom base plate, such that the top base plate and the bottom base plate are configured substantially parallel to each other along a vertical axis;
(c) a projection rotatably attached to the top base plate or the bottom base plate and lying along the vertical axis between the top base plate and bottom base plate, and rotatable about the longitudinal axis of the apparatus, such that the projection rotates from a “closed” position wherein the projection is fully contained within the circumference defined by the top base plate and the bottom base plate, to an “open” position wherein a portion of the projection protrudes outside of the circumference defined by the top base plate and the bottom base plate.

In certain embodiments, the present technology is directed to a method of stimulating a tactile response in a user, the method comprising providing an apparatus discussed herein to the user and contacting the skin of the user with the surface of the projection.

In certain embodiments, the present technology is directed to a system for customizing a sensory apparatus discussed herein based on a user's desires, the system comprising:
(a) obtaining, through one or more questions, information identifying a customer, and the customer's desired goals;
(b) suggesting customized features based on the information obtained in (a); and
(c) building the apparatus with the desired number of projections and the texture of each projection.

### BRIEF DESCRIPTION OF THE DRAWINGS

FIG. 1 shows an exemplary embodiment of a sensory apparatus as described herein.

FIG. 2A shows an exemplary embodiment of a sensory apparatus as described herein, including an upper portion that is configured to be held within an optional lower portion, an exemplary embodiment of which is shown in FIG. 2B.

FIG. 3 shows an exemplary embodiment of a sensory apparatus as described herein, with multiple projections or wings in “open”positions.

FIGS. 4A and 4B show an exemplary top base plate and an exemplary bottom base plate, respectively, in certain embodiments herein.

FIG. 5 shows an apparatus as described in certain embodiments herein, flipped over so that the view is from the bottom; as well as an optional stand separated from the apparatus.

### DETAILED DESCRIPTION

As used herein, all singular terms refer to both singular and plural values. That is, “a” or “an” or “the” all mean “one or more.” The term “or” as used herein means any one or more of the alternatives, including all of the alternatives.

As used herein, “substantially” means within 10% of a value. For example, “substantially orthogonal” or “substantially perpendicular” mean having an angle of within 10% of 90 degrees—that is, 81 degrees to 99 degrees.

As used herein, “user” means an individual who has a need or desire to utilize an apparatus, method or system herein, or someone who can benefit therapeutically from such apparatus, method or system. As used herein, “customer” means a person associated with a user (or can be the same person as the user), who has an interest in managing, or assisting in the management of, the user's neurodevelopmental condition, or who can input the user's preferences or information based on a diagnostic assessment of the user's condition; for example, a parent or other family member, a teacher, a therapist or other medical professional, or any other person knowledgeable about the user's condition or benefit of utilizing an apparatus, method or system herein.

As used herein, “therapeutic benefit” means management or treatment of any medical condition, including a neurodevelopmental condition. This can define any benefit, no matter how incremental and small, and does not require complete cure or amelioration of any condition.

Exemplary embodiments of the present disclosure can provide for a sensory apparatus having one or more extendable projections (also referred to herein as “wings”) that provide one or more textures. As used herein, “projection” or “wing” means an item movably attached to a portion of a top base plate or a bottom base plate, and at least partially rotatable about that portion, such that the projection or wing can be configured between a “closed” (not in use) position or an “open”(in use) position, depending on the needs of a user.

In certain embodiments, the projection or wing is attached such that it is rotatable about the portion of the top base plate or the bottom base plate in in a substantially perpendicular direction from the vertical axis of the apparatus. In certain embodiments, the one or more projections can rotate at least partially freely in a direction about the vertical axis of the apparatus. However, it is important to note that in other embodiments, the apparatus can be disposed substantially perpendicularly, such that the projections would be closer to vertical in such a case.

In certain embodiments, two wings can be aligned on substantially the same plane lying between the top base plate or bottom base plate, such that each wing, when in the closed position, takes up approximately half of the plane. In such a way, a user can simultaneously “open” both of the wings such that they protrude from different parts of the circumference whose edges are defined by the top cover and the bottom cover (or the top base plate and the bottom base plate). The embodiments herein also contemplate a configuration where only one wing takes up the entire circumference defined by the top base plate and the bottom base plate, as well as configuration where more than two wings can lie along the plane.

In certain embodiments, an apparatus herein has one or more further covering surfaces that cover a wing, such that when the wing is in a “closed” position, it is shielded in whole or in part by a covering surface. This can improve the ability of the user to store and transport an apparatus herein, and can be the difference between a portable apparatus and one that is not portable. In certain embodiments, a covering surface can be in the form of a bottom half or a receiving portion, as will be described later herein.

In certain embodiments, a projection or wing herein has an exterior surface. In certain embodiments, a projection or wing has a substantially flat top surface or bottom surface or both, such that it is easily tucked between the top base plate and bottom base plate when in the closed position, making for a streamlined apparatus.

Exemplary embodiments of the various apparatuses and systems herein will now be described with reference to the Figures. The following descriptions of the various embodiments is merely exemplary in nature and is in no way intended to limit the scope of the disclosure, its application, or uses.

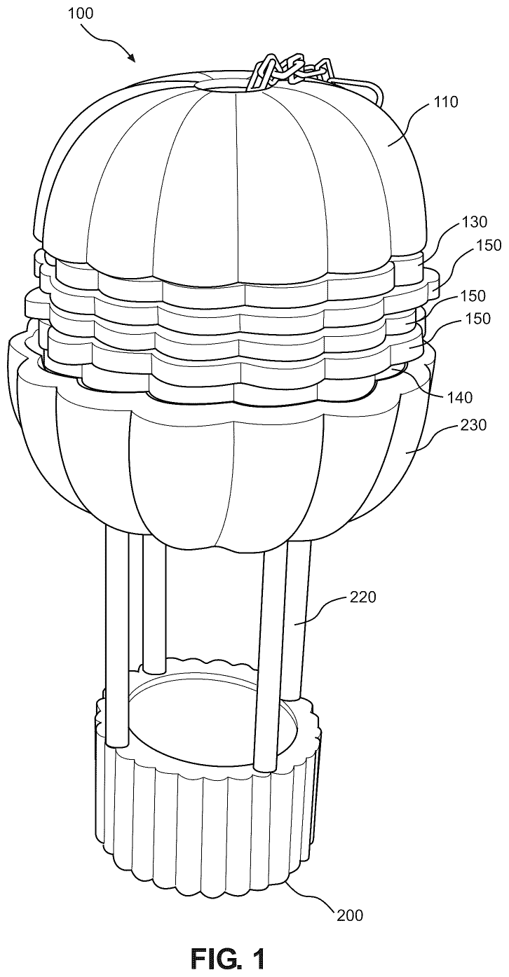

As shown in FIG. 1 through FIG. 5, in certain embodiments herein, a sensory apparatus 100 can be provided on a stand 200. The stand 200, which is optional, can include a base 210, one or more poles 220 provided on top of the base 210 and a receiving portion 230 provided on top of the poles to receive the sensory apparatus 100.

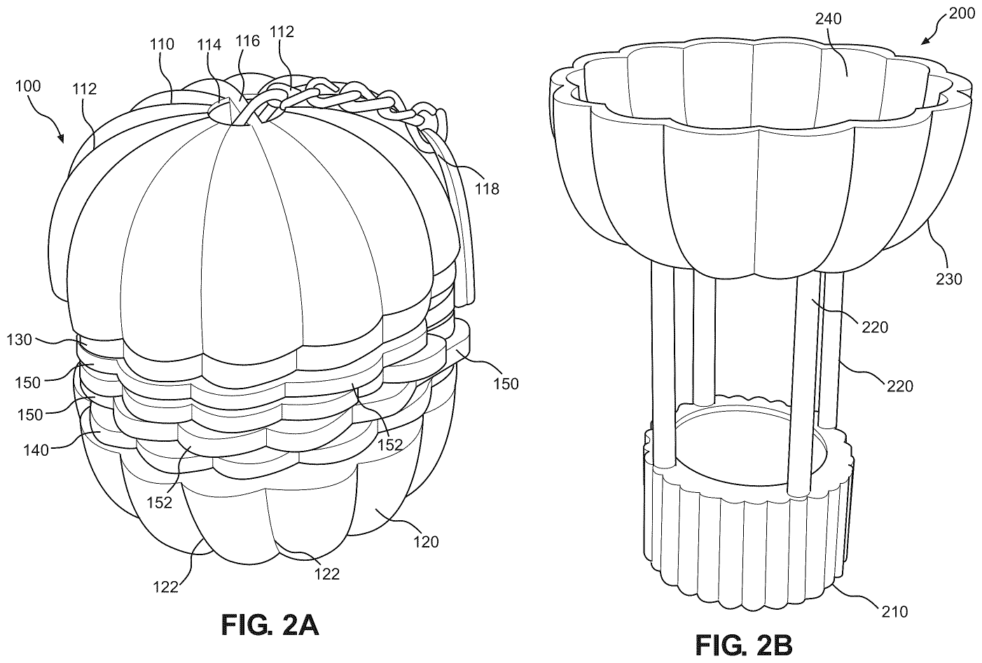

In certain embodiments, for example as shown in FIG. 2B, the receiving portion 230 can have a hollow interior 240 that generally conforms in part or in whole to the shape of the lower half of the sensory apparatus 100, as will be described below, in order to receive the sensory apparatus 100 and hold it securely. That is, the receiving portion can have a hollow interior configured to hold the apparatus through contact with the bottom cover.

In various embodiments, one or more poles 220 can be removably fit to the base 210 or receiving portion 230, or the one or more poles 220 can be integral with the base 210 or receiving portion 230.

In certain embodiments, the sensory apparatus 100 can have a top cover 110 and bottom cover 120, as seen in, for example, FIG. 2A. The bottom cover 120 can have a shape that conforms to the receiving portion 230 and size that is slightly smaller than the hollow interior 240 of the receiving portion 230 so that the sensory apparatus 100 fits within the stand 200 securely, such that, in various embodiments, the bottom cover 120 is wholly or partially invisible to a user when the sensory apparatus is fitted within the stand.

In certain embodiments, the top cover 110 can have a generally rounded shape e.g., a generally bulbous shape with optional ridges 112, and the bottom cover 120 can have a generally rounded shape, e.g., a generally bulbous shape with optional ridges 122, to form a textural element while giving an appearance of an object such as, e.g., a hot air balloon. As used herein, “generally rounded” means having a curved edge. As used herein, “generally bulbous”means having a shape that is rounded, protruding or bulging.

In certain embodiments, as shown in, for example, FIG. 2A, the top cover 110 can have a circular extrusion 114 cut out with a bar 116 in the top center of the top cover 110. In certain embodiments, a keychain 118 or other attachment device can be connected to the bar 116. As used herein, “keychain attachment” means an apparatus herein in the form of a toy or novelty device that can be attached to a keychain.

In certain embodiments, as shown in, for example, FIG. 1, a top base plate 130 can be provided that is connected to or integral with the top cover 110; or a bottom base plate 140 can be provided that is connected to or integral with the bottom cover 120. In certain embodiments, one or both of the top base plate and bottom base plate are attached to the top cover and bottom cover, respectively, by being glued, soldered or screwed in, snapped in, ultrasonically welded; or can be integral pieces with the top cover and bottom cover, respectively.

In various embodiments, the top cover and the bottom cover, or the top base plate and the bottom base plate, together define a circumference, and this sets the boundaries for the configuration of the one or more wings between them. As used herein, “circumference” does not require a perfect circle, but rather, a generally circular shape that defines the horizontal plane in which the one or more wings are held within the boundaries of the top cover and bottom cover (or the top base plate and the bottom base plate).

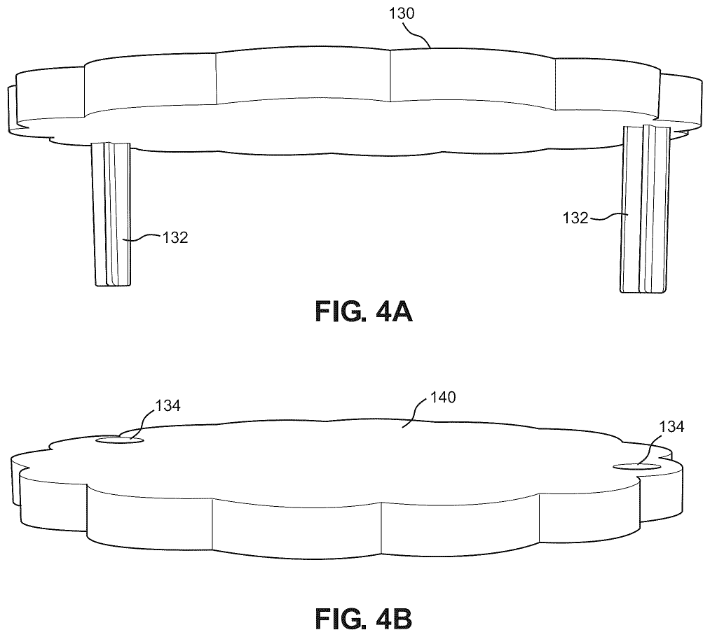

In certain embodiments, as shown in, for example, FIG. 4A, one or more studs 132, for example, spring loaded studs, can be provided between the top base plate 130 and the bottom base plate 140. In certain embodiments, the studs 132 can be connected to the top base plate 130 and fit within one or more slots 134 in the bottom base plate 140, as seen in, for example, FIG. 4B. In certain embodiments, a stud 132 can be snap fit to a slot 134. In certain embodiments, a stud 132 can be adjustable (e.g., spring loaded). This can be useful in several ways, for example, allowing for an adjustable distance between the top base plate 130 and bottom base plate 140 to fit multiple layers of wings as will be described below. For example, in certain embodiments, a stud such as a spring-loaded stud 132 can allow one or more wings to be inserted between the top base plate 130 and bottom base plate 140. Thus, a stud can be one about which a projection or wing rotates. The wings can rotate easily from an open position to a closed position, and stay snugly secured in place, but in certain embodiments, still be removable and switched out to per the user's preference.

In certain embodiments, one or more wings 150 can be provided between the top base plate 130 and the bottom base plate 140. In various embodiments, a wing 150 can be connected to one or more adjustable studs 132 such that the wing 150 is rotatable around the one or more studs 132; or can be removable such that different wings 150 can be installed between the top base plate 130 and bottom base plate 140. For example, in various embodiments, a wing 150 can be connected to a stud 132 using mechanisms such as an O-ring carabiner, a buckle or bearings; or multiple wings can be stackable over each other. In certain embodiments, two wings 150, or even more, can be provided in each layer.

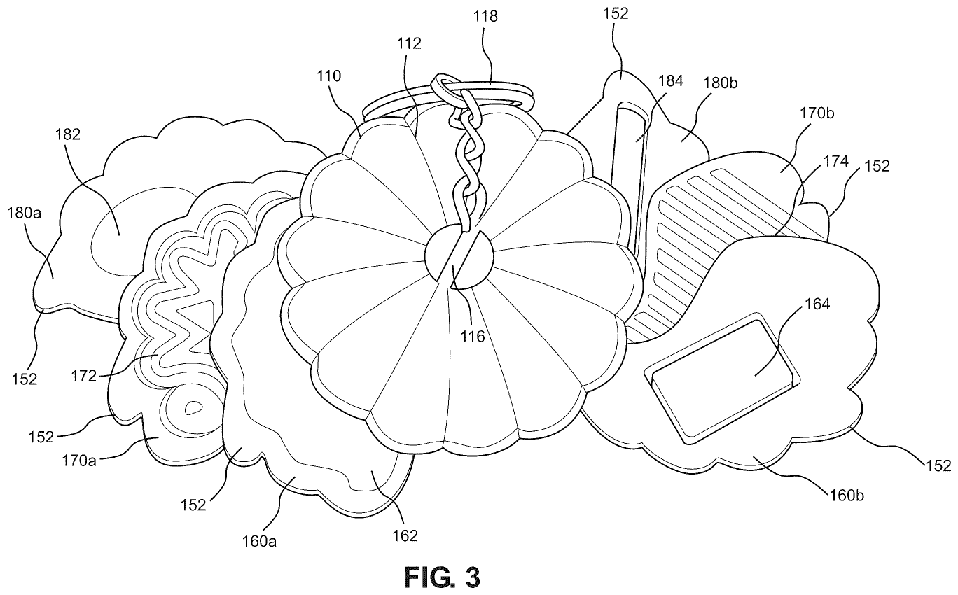

As shown in FIG. 2 and FIG. 3, in certain embodiments, three layers can be provided, each having two wings 150 (or more) in each layer. As shown in FIG. 1 and FIG. 2, in certain embodiments, the wings 150 can be held within a space between the top cover 110 and bottom cover 120; or a circumference defined by the top cover and the bottom cover (or the top base plate and the bottom base plate), such that when a wing is exposed in whole or part, this constitutes an “open” configuration, and when a wing is rotated to be fully held within the top cover or bottom cover, with no part of the edge of the wing protruding from an outside edge thereof, this constitutes a “closed” position.

In certain embodiments, a projection or wing herein has dimensions that generally make it flat on at least a top surface, such that a user can easily grasp it and feel its surface, thus gaining the benefits of an apparatus herein. For example, a projection or wing herein can have a generally “flat” configuration on at least one side, such that it is easily rotated between the “closed” and “open” positions, and that a user with basic fine motor skills can manipulate it; and in some embodiments, can practice and develop fine motor skills by using the apparatus.

In various embodiments, as shown in, for example, FIG. 3, a wing 150 can have one or more tabs 152 that allow a user to pull the wing 150 outward and rotate the wings 150 away from the top base plate 130 and bottom base plate 140, going from a “closed” position to an “open”position, or vice versa.

In certain embodiments, multiple wings can be provided that are provided stacked on each other to provide multiple accessories or attachments to the sensory apparatus. For example, in the embodiment shown in FIG. 3, wings 160a, 170a and 180a can be provided on a first side of the sensory apparatus 100, and wings 160b, 170b and 180b can be provided on a second side of the sensory apparatus 100. In this particular embodiment, wings 160a and 160b form a first layer, wings 170a and 170b form a second layer, and wings 180a and 180b form a third layer; although the ordering and configuration of the layers and the wings within each layer are not necessarily so limited, and can be any manner desirable to a user.

In various embodiments, each wing can have a different visual appearance or texture on a top surface, bottom surface, or edge thereof. For example, wing 160a can have a fabric or cloth 162 applied to a top surface thereof. ; wing 170a can have a design with ridges 172 on a top surface thereof; wing 180a can have a design with a deep groove 182 on a top surface thereof; wing 160b can have a flat revolving switch 164 on a top surface thereof; wing 170b can have a plurality of channels 174 on a top surface thereof; wing 180b can have a cutout 184 on a top surface thereof. In various embodiments, a wing can have a top surface and a bottom surface that are different, or the same, or in any configuration desirable to the user.

In various embodiments, a texture on a surface of a projection or wing can be an exterior surface of the projection or wing, and can be rough, ridged, protruding, pitted, or grooved. A texture can also be of varying degrees of roughness, or can feel smooth, abrasive, bumpy, corrugated, cratered, dented, embossed, encrusted, etched, feathery, fluffy, furry, grainy, gritty, hairy, hard, lumpy, malleable, padded, patterned, prickly, spiky, velvety, wiry, reflective, “reading” (e.g., a magnifying glass that groups words together to help focus), sliding, crinkly or the like.

The different visual appearances or textures on the surface of each wing can be any such visual appearances or textures that may be, without limitation, desirable to a user or useful for a therapeutic benefit.

In an exemplary embodiment, different wings can have a different texture, or two or more textures on a surface thereof. In certain embodiments, a texture may be applied to a top or bottom surface of a wing, or both; or a top or bottom surface of a single wing can each have a different texture. The wings are not limited to any type of texture, and a variety of textures, designs, grooves, ridges, patterns and fabrics can be applied to provide a variety of sensory tools. As non-limiting examples, tactile features (textures) such as fabrics and inlays can be provided, vestibular features such as revolving buttons or switches or flat buttons or switches, or visual features such as magnifying glass or one-line readers can be applied.

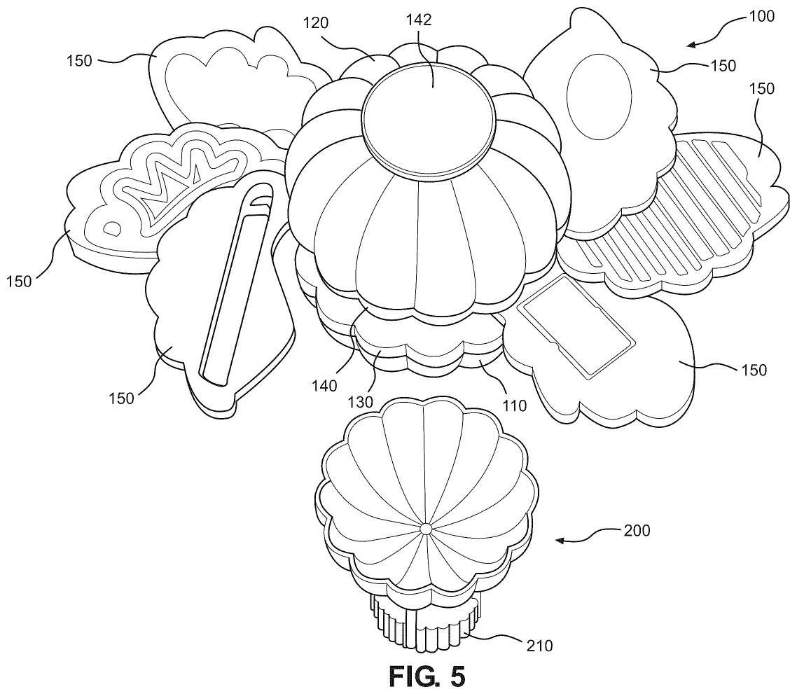

As shown in an exemplary embodiment in FIG. 5, in certain embodiments, the bottom cover 120 can have a disc 142 in or near the center of the bottom cover 120. The disc 142 can have a cylindrical tube shape that extends into the bottom cover 120 into a ball bearing to allow the bottom cover 120 to rotate around the disc 142. This can provide yet another sensory mechanism for user to play with, like fidget spinner. A wide variety of bottom covers 120 can be provided having different mechanisms such as the disc 142, in many categories, such as in a vestibular category, such as the disc 142. For example, one or more mechanisms in the auditory category could be provided in lieu of, or in addition to, the disc 142, such as an earplug holder or mini speaker, customized to user's needs. In various embodiments, one or more mechanisms in the gustatory and olfactory categories can be provided, such as a container to put things to satisfy such needs, such as mints or a scent that a user may prefers, that the user (or any other individual, e.g., a therapist, parent, or anyone assisting the user) can place within the container. Mechanisms in the visual or tactile categories can be provided, that may be similar to the bottom cover 120 that has ridges to form a textural element.

In certain embodiments, the sensory apparatus 100 can appear similar to an object such as a hot air balloon. The imagery of a hot air balloon can evoke a sense of nerve easing peace and rest. However, it is important to note that in other embodiments, an apparatus herein comprises only the generally round part defined by the top cover and the bottom cover, and does not include the base portion. In such embodiments, the appearance of the apparatus will be more like an “egg” than a hot air balloon; or more like a balloon without the attached basket.

In various embodiments, a color scheme in one or more shades of blue, such as cobalt and light blue can be used to show, e.g., sensory processing disorder awareness.

Such colors can also represent calmness and serenity in the mind within color psychology.

However, any color scheme of an apparatus herein can be any of a variety of colors desirable to a user, and in various embodiments, the sensory apparatus 100 can be customized to any color or colors the customer prefers.

In various embodiments, an apparatus herein provides an advantage of being portable. As used herein, “portable” means easily transported or carried by a user without the need for specialized equipment or separation of parts. In certain embodiments herein, an apparatus according to the present technology is made of lightweight material, can be configured such that when the one or more projections is in the “closed” position, the apparatus can be easily held in just one hand and transported with minimal effort on the part of the user.

In certain embodiments, the present technology is directed to a consumer product comprising a sensory apparatus with various customizable parts that can be self-assembled-for example, including one or more of the following:
Case with a keychain;
Spin case (including a ball bearing)
Earplug holder case (opening with a hinge)
Holder to transport the apparatus, configured to hold the wings together during transport;
1 metal (or strong plastic) pins with snap fit or threaded ends;
1 base plate to attach to keychain;
1 base plate to secure ball bearing;
1 base plate to secure earplugs;
Material: hard plastic.

In various embodiments, the projections or wings can be put into different categories. For example, they can be textured (e.g., engraved), made of additional material and fit to size, e.g., by molding or cutting (for example, soft, squishy, or rough). In other embodiments, they can be classified as “activity” wings-e.g., embedded cuts or through the wing, moving wings (for example, with switches or slides). In various embodiments, a product herein can include 8 to 12 wings, which the user can customize with regard to order in the apparatus.

In certain embodiments, the technology herein is directed to methods comprising providing an apparatus herein to a user and contacting the skin of the user with the surface of the projection. For example, a projection herein can have an exterior surface comprising a texture that stimulates the tactile response when contacted with the skin of the user. In certain embodiments, the “skin” of the user refers to any tactile surface on the user's body that has receptors that transmit the sensation of touch to the user-for example, hands, including palms and fingers, but also feet, faces, or any part of the arm.

In certain embodiments, the technology herein is directed to a system for customizing a sensory apparatus based on a user's needs. For example, a website can be provided where a customer can be asked to complete a questionnaire (e.g., medical diagnostic questionnaire, or questionnaire regarding any therapeutic goal or user preference) to help determine if the person is “hyposensitive” (i.e., seeks out sensory stimuli) or “hypersensitive” (i.e., avoids sensory stimuli). These “yes” or “no” questions include but are not limited to questions such as those below, and can be provided, for example, as follows, in no particular order:

In certain embodiments, the technology herein is directed to a system for customizing a sensory apparatus as discussed herein, to specifications that are beneficial for use by a user. That is, in certain embodiments, once the customer completes a questionnaire, a system herein is configured to do one or more of the following: tally the answers and generate results, provide an explanation of these results, and recommend customized features, e.g., cover and wing module options, in an output, e.g., on a user interface such as a website or application. In certain embodiments, after the customer chooses a bottom cover and at least 2 wings from the selection and purchases it, a receipt can be provided to the customer with the results and its explanation. Then, a fully personalized sensory apparatus 100 can be shipped to the customer.

In certain embodiments, an in-store or point of sale option can also be provided with a questionnaire in written form or electronic form, e.g., provided on a table, provided in a computer interface or in an electronic application, where the recommended options will be provided to the customer at the store and discussed with a store representative or emailed to the customer directly.

Among the information that can be gathered from the user are: information identifying the user (for example, name, age, sex, and neurodevelopmental information or diagnoses); specific likes and dislikes (for example, known textures, visual, aural or olfactory items that calm versus those that trigger or cause anxiety, or phobias); and information about the user's goals (for example, amelioration or management of sensory issues; distraction from stressful stimuli or environmental triggers; calming or decrease of stress). In various embodiments, a system herein can then suggest details such as textures, configuration of the wings, or stimuli to avoid or to include in the customized apparatus.

Various other considerations can also be addressed in the exemplary applications described according to the exemplary embodiments of the present disclosure. For example, in certain embodiments, various numbers of wings with different textures can be provided. Various sizes of the sensory apparatus can also be provided.

In various embodiments, a sensory apparatus herein and parts (e.g., any part of the wings including the textures and surfaces), can be made in whole or in part of various materials, such as plastic, wood, metal, fabric, leather, natural or synthetic rubber, minerals such as rock or stone, a mirror or reflective material, feathers, fur, scales, reptile skin, bristles, or any composites thereof. The sensory apparatus can be portable and lightweight such that it is easily transportable by the user and can be used in a variety of settings without a significant amount of advanced planning or inconvenient transport.

In certain embodiments, a sensory apparatus herein can be a customizable fidget multitool designed to mitigate sensory overload for children or adults with any of a variety of neurodevelopmental conditions, including but not limited to sensory processing disorder (SPD). For example, a sensory apparatus herein can be in the form of a keychain attachment, that a user can carry around and use during the normal course of a day-for example, while commuting to school or work, riding public transportation, sitting and waiting, or any time the user feels a need to engage with the sensory apparatus.

The various embodiments herein, whether directed to an apparatus, method, or system, can have as an advantage the stimulation of a desired tactile response in a user. As used herein, “tactile response” means any response by an individual to tactile stimuli, including a message related to any of the following: pressure, vibration, texture, temperature, pain, and position, including position of limbs. Individuals with neurodevelopmental conditions can benefit from the apparatuses, methods, and systems herein. In various embodiments, the existence and quality of a tactile response can be reported by a user or observed by a third party.

Although the present technology has been described in relation to embodiments thereof, these embodiments and examples are merely exemplary and not intended to be limiting. The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure.

1. An apparatus configured to stimulate a tactile response in a user, the apparatus comprising:
(a) a top cover having a generally rounded top surface and an opposite surface connected to or integral with a top base plate;
(b) a bottom cover disposed opposite the top cover, having a generally rounded bottom surface and an opposite surface connected to or integral with a bottom base plate, such that the top base plate and the bottom base plate are configured substantially parallel to each other along a vertical axis;
(c) a projection rotatably attached to the top base plate or the bottom base plate and lying along the vertical axis between the top base plate and bottom base plate, and rotatable about the longitudinal axis of the apparatus, such that the projection rotates from a “closed” position wherein the projection is fully contained within the circumference defined by the top base plate and the bottom base plate, to an “open” position wherein a portion of the projection protrudes outside of the circumference defined by the top base plate and the bottom base plate.

2. The apparatus of claim 1, wherein the projection has an exterior surface, the exterior surface comprising a texture that stimulates the tactile response when contacted with the skin of the user.

3. The apparatus of claim 1, wherein one or both of the top base plate or bottom base plate comprises a stud about which the projection rotates.

4. The apparatus of claim 3, wherein one or both of the top base plate or bottom base plate comprises a slot into which the stud fits.

5. The apparatus of claim 1, further comprising a base, the base including a receiving portion having a hollow interior configured to hold the apparatus through contact with the bottom cover.

6. The apparatus of claim 1, wherein one or both of the top cover or the bottom cover has a generally bulbous shape.

7. The apparatus of claim 1, wherein an exterior surface of the projection is rough, ridged, protruding, pitted, or grooved.

8. The apparatus of claim 1, in the form of a keychain attachment.

9. A method of stimulating a tactile response in a user, the method comprising providing an apparatus of claim 1 to the user and contacting the skin of the user with the surface of the projection.

10. The method of claim 9, wherein the tactile response results in a therapeutic benefit to the user.

11. A system for customizing a sensory apparatus of claim 1 based on a user's desires, the system comprising:
(a) obtaining, through one or more questions, information identifying a customer, and the customer's desired goals;
(b) suggesting customized features based on the information obtained in (a); and
(c) building the apparatus with the desired number of projections and the texture of each projection.
